(19) United States
(12) Patent Application Publication
(10) Pub. No.: US 2010/0055161 A1
(43) Pub. Date: Mar. 4, 2010

(54) HYDROGEL FACE MASK FOR DELIVERING SKIN CARE AGENTS

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/199,751

(22) Filed: Aug. 27, 2008

(51) Int. Cl. A61K 9/70; A61K 33/04; A61P 17/00

(52) U.S. Cl. 424/449; 424/617

(57) ABSTRACT

Skin care can be provided by the skin care devices and compositions described herein. Such a skin care device can be configured to include a biocompatible hydrogel layer including one or more treatment agents, and an associated backing layer associated with the hydrogel layer.

### BACKGROUND

The skin is the outer covering of living tissue of an animal, and includes multiple layers of epithelial tissues. The skin is the first line of protection for the underlying tissues, vasculature, muscles, bones, ligaments, internal organs, and the like. The structure of skin provides protection from the outside environment; mainly as a barrier against pathogens and the sun. Additionally, the skin plays important roles in providing insulation, temperature regulation, sensation, synthesis of vitamin D, and protection of vitamin B folates.

When damaged, skin becomes less efficient in functioning properly, which can lead an individual to be more susceptible to pathogens, sun damage, punctures, and abrasions. Skin damage can arise from being physically injured, exposure to certain conditions, and from natural and accelerated aging. Severely damaged skin will try to heal by forming scar tissue that is often discolored and/or depigmented. Additionally, scar tissue can be unsightly, and can cause emotional distress when present on a visible portion of skin, such as on the face. As a result, various therapies and treatments have been sought to protect skin from being damaged as well as to treat skin that has been damaged to reduce scar formation. Also, many therapies and treatments have been prepared in an attempt to reduce or inhibit skin aging.

The various therapies and treatments have been provided in many different usage forms, such as for internal or external application. Therapies and treatments for external application have been formulated as gels, lotions, sprays, and the like. However, many of these formulations have shortcomings, such as dripping or unwanted spreading of the therapeutic agent, and therefore improved skin therapies and treatments continue to be researched and developed.

### BRIEF SUMMARY

Generally, skin care can be provided by the devices and compositions described herein in order to treat and/or prevent a skin condition, disease, disorder, or symptoms thereof. Also, the devices and compositions described herein can provide general skin care agents to the skin for maintenance of skin form and function. The devices and compositions can be applied to the skin in order to provide the prophylactic, therapy, or maintenance for enhanced skin care.

In one embodiment, a device for delivering one or more treatment agents to skin can include: a biocompatible hydrogel layer; one or more treatment agents included in the hydrogel layer; and a backing layer associated or in contact with the hydrogel layer. For example, the one or more treatment agents can be configured for treatment and/or prevention of at least one of acne, sunspots, skin cancer, wrinkles, dry skin, eczema, edema, psoriasis, rashes, shingles, infection, inflammation, itching, seborrheic dermatitis, atopic dermatitis, warts, rosacea, fungal infections, herpes, razor burn, mastocytosis, intertrigo, pityriasis rosea, lichen planus, hidradentis, symptoms thereof, combinations thereof, and the like.

In one embodiment, the hydrogel layer can have a shape and size for application to a desired portion of skin of a subject. In one option, a desired portion of skin can be selected from the group consisting of a forehead, nose, cheek, lips, face, neck, shoulder, arm pit, arm, hand, finger, abdomen, chest, stomach, back, buttocks, genitals, legs, knee, feet, toes, and combinations thereof. In one option, the shape and size can be configured for application to a face of the subject.

In one embodiment, a device for delivering one or more treatment agents can be configured or characterized by at least one of the following: the biocompatible hydrogel layer is configured to retain structural integrity when on or in contact with skin; the one or more treatment agents include a compound or composition configured to provide a skin treatment; the backing layer is configured to retain structural integrity of the hydrogel layer when the hydrogel layer is the skin; the one or more treatment agents are homogeneously dispersed within the hydrogel layer; the one or more treatment agents are included in a concentration gradient within the hydrogel layer; the one or more treatment agents are included as depots within the hydrogel layer; the backing layer is aqueous liquid impermeable; the backing layer is substantially hydrophobic; or the backing layer is paper.

In one embodiment, the device can further include at least one of the following: an adhesive layer between and coupling the backing layer to the hydrogel layer; a skin penetration enhancer included in the hydrogel layer, said skin penetration enhancer being configured to enhance penetration of the one or more treatment agents into skin; a peelable layer included on, associated with, or contacting the hydrogel layer; a peelable layer on the hydrogel layer so as to protect the hydrogel layer from drying during storage; or at least one opening in the hydrogel layer and backing layer that corresponds with at least one of an eye, nose, nostril, lips, mouth, or combination thereof.

In one embodiment, a method of providing one or more treatment agents to skin of a subject can include: providing a device for delivering one or more treatment agents to skin; and applying the hydrogel layer to the skin of the subject. The hydrogel can be retained on the skin while the one or more treatment agents is applied to the skin. In the instance that the device includes a peelable layer on the hydrogel layer, the method can include peeling the peelable layer from the hydrogel layer so as to expose a surface of the hydrogel for application to the skin. Also, in embodiments where the backing layer has a surface area larger than the hydrogel layer, the peelable layer can also be included on or in contact with the hydrogel side of the backing layer.

In one embodiment, a method of manufacturing a device for delivering one or more treatment agents to skin can include: preparing a biocompatible hydrogel or hydrogel-precursor composition; introducing one or more treatment agents into the hydrogel or hydrogel-precursor composition; and coupling a backing material to the hydrogel or hydrogel-precursor composition so as to form a hydrogel layer associated with or in contact with a backing layer.

In one embodiment, a method of manufacturing a device for delivering one or more treatment agents to skin can include: configuring the hydrogel layer to retain structural integrity when in contact with skin; configuring one or more treatment agents to include a compound or composition that provides a skin treatment; configuring the backing layer to retain structural integrity of the hydrogel layer when the hydrogel layer is in contact with the skin; homogeneously dispersing the one or more treatment agents into the hydrogel or hydrogel-precursor composition; distributing the one or more treatment agents into the hydrogel layer in a concentration gradient; or configuring the one or more treatment agents to be introduced into the hydrogel layer as depots.

In one embodiment, the method of manufacturing a device for delivering one or more treatment agents to skin can further include at least one of the following: providing an adhesive layer between the backing layer and the hydrogel layer (e.g. to adhere the two layers together); introducing a skin penetration enhancer into the hydrogel layer, said skin penetration enhancer being configured to enhance penetration of the one or more treatment agents into skin; applying a peelable layer onto the hydrogel layer (e.g., to protect the hydrogel layer from drying during storage).

These and other embodiments and features of the skin care device and compositions will become more fully apparent from the following description and appended claims, or may be learned by the practice of skin care as set forth hereinafter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

To further clarify the above and other advantages and features of the skin care device and compositions, an illustrative description of the skin care device will be rendered by reference to the appended drawings. It is appreciated that these drawings depict only illustrated embodiments of the skin care device and are therefore not to be considered limiting of its scope.

FIG. 1A is a schematic representation of an illustrative embodiment of a skin care device in the form of a face mask having a hydrogel layer for providing skin care.

FIG. 1B is a schematic representation of an illustrative embodiment skin care device for delivering a therapeutic agent to portions of skin.

FIGS. 2A-2C are schematic illustrations of the different layers of an illustrative embodiment of a skin care device having a hydrogel layer for providing skin care.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Generally, the skin care devices and compositions described herein can be used to treat and/or prevent a skin condition, disease, disorder, or symptoms thereof. Accordingly, the skin care devices and compositions can be configured to provide a therapeutic agent to the skin so as to provide a treatment or prophylactic effect. Also, the skin care devices and compositions can also provide general skin care agents to the skin for maintenance of skin form and function.

A skin care device can be configured for use in delivering one or more skin care agents (e.g., cosmeceuticals, pharmaceutical, nutraceutical, or general skin nutrients) to skin. Such a skin care device can include a biocompatible hydrogel that contains one or more treatment agents. The hydrogel can be any hydrogel that can be applied to skin. In some instances, the hydrogel can be compatible with the skin, and thereby is biocompatible. The treatment agents can be any agent that can provide general skin care as well as treatment agents that provide therapeutic and/or prophylactic effects. The skin care device can also include a backing layer associated with the hydrogel layer. The backing layer can be included on the skin care device to provide a substrate to the hydrogel for ease of handling as well as other functions.

The skin care device can be configured for the treatment and/or prevention of at least one of acne, sunspots, skin cancer, wrinkles, dry skin, eczema, edema, psoriasis, rashes, shingles, infection, inflammation, itching, seborrheic dermatitis, atopic dermatitis, warts, rosacea, fungal infections, herpes, razor burn, mastocytosis, intertrigo, pityriasis rosea, lichen planus, hidradentis, symptoms thereof, combinations thereof, and the like. Also, the skin care device can be configured to provide general skin maintenance such as by providing skin lotion components or components of dermatological compositions.

The hydrogel layer can be configured with a shape and/or size for application to a desired portion of skin of a subject. For example, the hydrogel layer can be shaped and sized to correspond with a desired portion of skin to receive the skin care treatment. Such a desired portion of skin can be selected from the group consisting of a forehead, nose, cheek, lips, face, neck, shoulder, arm pit, arm, hand, finger, abdomen, chest, stomach, back, buttocks, genitals, legs, knee, feet, toes, combinations thereof, and the like. Thus, the hydrogel layer, and the device in general, can be shaped and sized to be applied to any portion of skin on a subject's body.

One common area for receiving skin care is the face. The face is an important portion of skin to maintain or treat so that it is in good condition. For the face, the skin care device can be configured, for example without limitation, into a mask. As such, the hydrogel layer, and device in general, can be configured to have the shape and size of a mask or other shape or configuration for application to the face or a portion of the face of a subject.

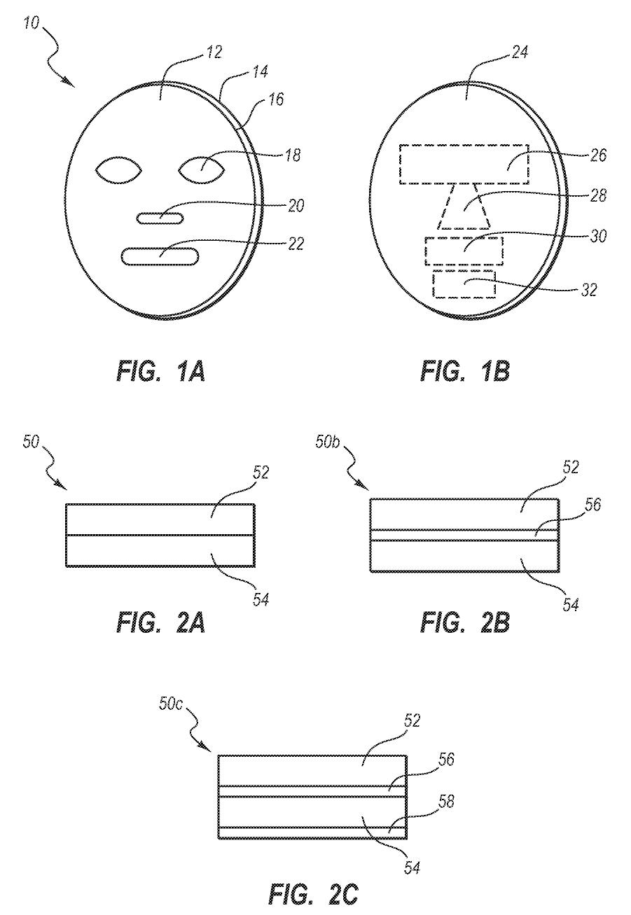

FIG. 1A is a schematic representation of a skin care device in the shape of a mask 10 that can provide skin care agents to the face of a subject. The mask 10 can be configured to include a backing layer 12 and a hydrogel layer 14, which can be coupled together at an interface 16. As shown, the mask 10 can include facial features, such as eye holes 18, a nose hole 20, and a mouth hole 22. Accordingly, the mask 10 can include at least one opening in the hydrogel layer 14 and/or backing layer 12 that corresponds with at least one of an eye, nose, nostril, lips, mouth, or combination thereof. The mask 10 can have a generic facial shape to conform with a wide rage of facial shapes and sizes. Alternatively, the mask 10 can be tailored to conform with specific facial shapes and sizes, such as round, oval, square, large, small, and other shapes or sizes.

FIG. 1B is a schematic representation of a skin care device 24 that has different regions with optionally different skin care configurations. The different regions can take into account tailored skin care treatments for specific portions of the skin. This can arise from one portion of skin needing a different care regimen compared to another portion. As shown, in a non-limiting example, a forehead portion 26 can be configured with skin care components and dimensions that are suitable for the forehead of a subject. A nose portion 28 can be configured with skin care components and dimensions that are suitable for the nose of a subject. A lip portion 30 can be configured with skin care components and dimensions that are suitable for the lips of a subject. A chin portion 32 can be configured with skin care components and dimensions that are suitable for the chin of a subject. Alternatively, separate skin care devices can be prepared in accordance with the portions shown in FIG. 1B. Similarly, the skin care devices can be configured for any portion of skin on a subject.

FIG. 2A is a schematic representation of an embodiment of the different layers that can be employed in a skin care device 50. As shown, the skin care device 50 can include a backing layer 52 and a hydrogel layer 54. The hydrogel layer 54 can include a therapeutic agent for providing skin care. Thy hydrogel layer 54 can be prepared and coupled to the backing layer 52, or vice versa. The hydrogel layer 54 can be directly or indirectly coupled to the backing layer 52. Examples of direct coupling include the hydrogel layer 54 having a material with sufficient adhesion characteristics in comparison to the backing layer material to adhere the hydrogel layer 54 to the backing layer 52. Indirect coupling can include the use of adhesives and/or other layers between the hydrogel layer 54 and the backing layer 52.

FIG. 2B is a schematic representation of an embodiment of layers of a skin care device 50 that includes an adhesive layer 56 included between the hydrogel layer 54 and the backing layer 52. The adhesive layer 56 can be prepared from any type of adhesive that can functionally couple the hydrogel layer 54 to the backing layer 52. The composition of the hydrogel layer 54 and/or the backing layer 52 can be determinative for the type of adhesive in the adhesive layer 56 because the adhesive is adhered to these two layers so as to couple the layers together. Pressure sensitive adhesives based on silicone adhesive, polyisobutylenes, polyacrylates, and polymethacrylates are non-limiting examples of suitable adhesives. Other general adhesives can also be used.

FIG. 2C is a schematic representation of an embodiment of layers of a skin care device 50 that is configured to have enhanced storage stability. As shown, the skin care device 50 includes a backing layer 52 coupled to a hydrogel layer 54 via an adhesive layer 56 as shown in FIG. 2B. Additionally, the skin care device 50 includes a peelable layer 58 on the hydrogel layer 54. The peelable layer 58 can be configured to cover the hydrogel layer 54 in a manner that protects the hydrogel material. For example, the peelable layer 54 from drying out or otherwise degrading during the storage of the skin care device 50. The peelable layer 58 can be prepared from a polymeric material as well as paper-based materials and combinations thereof.

The hydrogel layer can have a surface area that is associated with the backing layer, wherein the surface area can be larger, smaller, or about the same as the surface area of the backing layer. When the surface area of the backing layer is larger than the surface area of the hydrogel layer, the overhanging portion or surface of the backing layer can include at least one of the following; a skin adhesive, a peelable layer, the peelable layer that is also on the hydrogel layer; or the like. When the surface area of the backing layer is smaller than the surface of the hydrogel layer, the overhanging portion of surface of the hydrogel layer can include at least one of the following: nothing; a peelable layer; a portion of the peelable layer that also covers the skin contacting side of the hydrogel, and the like.

In one embodiment, the biocompatible hydrogel layer can be configured to retain structural integrity when in contact with skin. Hydrogels can be prepared with different structural properties depending on the additive components included in the hydrogel, as well as the polymeric components. For example, crosslinking agents or processes that induce crosslinking can be employed to provide increased structural integrity to the hydrogel. Also, the hydrogel polymers can be sufficiently cohesive so that the hydrogel has increased structural integrity. Additionally, rheology modifying agents can be used to modulate the strength of the hydrogel, which thereby increases the structural integrity. By modulating the hydrogel composition and method of manufacture, a hydrogel with sufficient structural integrity can be achieved for the different purposes and uses as described herein. For example, gels can be processed with different parameters to obtain more solid hydrogels that are similar in strength and flexibility to, for example, gelatins that have enough strength to stand alone without the aide of a supporting member. Such configurations of the hydrogel material can provide a hydrogel layer that can stand alone on a patient's skin without a supporting member. In instances where the hydrogel layer can be used alone, the backing can be utilized for purposes other than structural support, as described in more detail herein.

In one embodiment, the backing layer is configured to retain structural integrity of the hydrogel layer. In a non-limiting example, the backing layer can provide structural integrity to the hydrogel layer when in storage or the hydrogel layer is in contact with the skin. In some instances, a hydrogel can be gelatinous and lacks sufficient structural integrity to stand alone without supplemental support. As such, the backing layer can provide such support by having structural soundness that allows for bending and flexing without breakage or cracking. The ability to modify the structural strength of the backing layer to support a gelatinous hydrogel layer can allow for the hydrogel to be configured without substantial structural strength because it can reside on the backing. The transdermal drug delivery arts have utilized backing layers to provide structural stability to medical devices, and such backing layers can be applied to the skin care device to hold and retain a hydrogel layer in a skin care device.

In one embodiment, the backing layer can be configured to be an aqueous liquid impermeable layer, which can be referred to as being water resistant. Since the hydrogel layer can be prepared to include a substantial amount of water so as to hydrate the polymer into a gel form, the backing layer being water resistant can aid in maintaining the gel form of the hydrogel. Also, the water resistant backing can retain water in the hydrogel during storage and use so that the hydrogel does not dry out. The water resistant backing layer can be prepared from a hydrophobic material, such as but not limited to, a hydrophobic polymer, such as, without limitation, polyethylenes, polystyrenes, polyurethanes, polyolifins, and the like. Foils, such as Tyvek and others, can also be used. In addition, the water resistant backing layer can be configured to keep additional water, such as from sweat or other sources, from entering into the hydrogel in a negative manner that compromises the composition of the hydrogel. Similar to backing layers of transdermal devices providing structural integrity, many transdermal devices include water resistant backing layers and such water resistant backing layers can be applied to the skin care device.

In one embodiment, the backing layer of the skin care device described herein can be prepared from a paper or a paper-like material (e.g., fabrics, meshes, Paper or paper-like materials have been used in skin care applications for skin care treatments. For example, Biore facial masks, which are paper or paper-like have been used to provide a therapy to facial skin, such as to provide ingredients or aid in the removal of blackheads or acne from the skin. The paper of the skin care device can be coated with polymers or other agents as well as be applied to the hydrogel. In some instances, the hydrogel is applied to the paper backing layer so that the paper backing can function as described herein. Also, the paper backing can be configured so that some moisture penetrates the paper into the hydrogel to increase the water content of the hydrogel layer, which can be beneficial in some instances, such as for maintaining or increasing the moisture content of the hydrogel.

The hydrogel layer can be configured with the one or more treatment agents being homogeneously or substantially homogeneously dispersed therein to provide a homogenous or substantially homogenous composition. Homogenous layers can be beneficial because the treatment agent is uniformly distributed in the hydrogel such that delivery can be substantially uniform so as to avoid burst effects, lag, or other unfavorable delivery kinetics. The homogeneous hydrogel layer can be prepared by mixing the hydrogel with the treatment agent until substantially uniform in distribution of the components is achieved. Also, homogeneous distribution can be achieved by hydrating the hydrogel layer in a solution, such as but not limited to water, aqueous buffers, salines, alcohols, and the like that hydrate hydrogels, that includes the treatment agent until a steady state equilibrium is obtained where the treatment agent is evenly partitioned throughout the hydrogel.

The hydrogel layer can also be configured with the one or more treatment agents being included in the hydrogel layer in a concentration gradient. Depending on the desired release profile of the treatment agent, the concentration gradient can be more concentrated at the backing layer side of the hydrogel or more concentrated at the skin application side of the hydrogel layer. Concentration gradients can be useful for providing a delivery rate that is appropriate for the treatment agent being delivered. In some instances, the higher concentration at the skin application side of the hydrogel can allow for a faster flux in the beginning of the treatment so that an increased amount of treatment agent is initially provided to the skin and the following flux maintains at least a suitable level of the treatment agent. On the other hand, a certain treatment agent may have some negative effects at high concentrations so that a slower delivery profile to the skin is desired, and which can be obtained by the higher concentration in the hydrogel being at the backing layer. The concentration gradients can be achieved by selective incorporation of the treatment agent into the hydrogel, typically by selective absorption into one side (e.g., backing layer portion or skin portion) or the other. For example, a concentration gradient can have a first concentration on one side of the hydrogel and then a second concentration on the opposite side of the hydrogel, wherein the second concentration is double, triple, quadruple, or other factor greater that the first concentration.

The hydrogel layer can also be configured with the one or more treatment agents being included in the hydrogel layer as depots or reservoirs. The depots can include the treatment agent being agglomerated together with or without agglomerating adjuvants within the hydrogel material. For example, the depot can be a solid, emulsion, liquid, paste, gel, or the like. In some instances, the depots can be formed similarly to capsules of the treatment agent and be included within the hydrogel. Such depots can be formed during preparation of the hydrogel layer or can be provided into the hydrogel layer after being prepared.

In one embodiment, the hydrogel layer can include a skin penetration enhancer that enhances the delivery and/or penetration of the treatment agent to and/or into the skin. Penetration enhancers can be selected based on the properties of the hydrogel layer, skin, and the treatment agent being delivered. Additionally, the depth of penetration of the treatment agent can be a parameter for selection of a penetration enhancer. Penetration enhancers are well known to be used in transdermal devices, and can be employed in the skin care device. Non-limiting examples of penetration enhancers can include sulphoxides, dimethylsulphoxide (DMSO), azones such as laurocapram, pyrrolidones such as 2-pyrrolidone, alcohols, ethanol, alkanols, decanol, glycols, propylene glycol, surfactants, terpenes, and the like.

As described, the hydrogel can be prepared from any biocompatible polymeric composition that forms a gel or gelatinous composition and that can be applied to the skin of a subject to deliver the treatment agents as described herein. As such, the hydrogel can be configured into a gel that is a solid, jelly-like material formed from a polymeric and/or colloidal solution. By weight, gels are mostly liquid, yet behave like solids due to the addition of a gelling agent, polymer, crosslinker, rheology modifying agent, gelling agent, or other that aids in forming a gel. The hydrogel includes a solid network of materials that span the volume of a liquid medium so as to form the gelatinous material. The network can be composed of a wide variety of materials, including but not limited to particles, polymers and proteins. Non-limiting examples are provided below.

The hydrogel can include superabsorbent natural or synthetic polymers that can absorb substantial amounts of water. The hydrogels can possess a degree of flexibility very similar to natural tissue, due to their significant water content. Many hydrogels display thixotropy by becoming more fluidic when agitated, but resolidify when resting. In general, gels appear to be solid, jelly-like materials that can be exemplified by the consistency of jellyfish.

The hydrogel can include one or more gel-forming hydrocolloids. The term “gel-forming hydrocolloid” refers to a polymeric material that absorbs water to form a coherent gel under physiological conditions of temperature and pH. Preferably, the hydrocolloid absorbs at least 100% w/w, more preferably at least 300% w/w of water on immersion at 25° C. for 24 hours. The hydrogel can be water-swellable, but not water-soluble.

The hydrogel can include cationic polymers, which are positively charged polymers. The positive charges of the polymers can prevent the formation of coiled polymers, which allows the polymers contribute more to viscosity by having a stretched state. The stretched-out polymer can take up more space than a coiled polymer, and thereby resists the flow of solvent molecules around it. Cationic polymers are a main functional component of many gels, because the positive charged polymers also bind the negatively charged amino acids and lipids that are present on the surface of skin. This allows for the hydrogel to have some bioadhesive properties.

The hydrogel can also include anionic polymers, neutral polymers, as well as polymers that include hydrophilic and/or hydrophobic moieties. Such polymers are commonly used in preparing hydrogels. The combination of anionic polymers and cationic polymers can provide for ionic crosslinking that can aid in providing structural strength to the hydrogel. The use of hydrophilic and hydrophobic polymers can also create areas in the hydrogel that can be configured for retaining and releasing the treatment agent, especially when the treatment agent is hydrophobic.

Examples of materials that can be used to prepare hydrogels include polymers, copolymers, and monomers of: vinylpyrrolidones, methacrylamides, methacrylates, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, polysaccharides, alginates, dextrans, celluloses, modified celluloses, oxidized celluloses, chitosans, chitins, guars, carrageenans, hyaluronic acids, inulin, starches, modified starches, agarose, methylcelluloses, plant gums, hylaronans, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI {itaconic acid-bis(1-propyl sulfonizacid-3) ester di-potassium salt}, itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, combinations thereof, and the like.

In one embodiment, the hydrogel can be conditioned with a plasticizer. The plasticizer is selected from the group consisting of glycerol, propylene glycol, polyethylene glycol, polypropylene glycol, sorbitol, other glycols and ether glycols such as mono- or diethers of polyalkylene glycol, mono- or diester polyalkylene glycols, polyethylene glycols glycolates, ethylene glycol, diethylene glycol, triethylene glycol, propylene glycol dipelargonate and polypropylene glycol glycerol, sorbitan esters, esters of citric and tartaric acid, imidazoline derived amphoteric surfactants, lactams, amides, polyamides, quaternary ammonium compounds, esters such phthalates, adipates, stearates, palmitates, sebacates, or myristates, and combinations thereof. diisopropyl adipate, phthalates and diethyl sebacate; hydrocarbons such as liquid paraffin; ethoxylated stearyl alcohol, glycerol esters, isopropyl myristate, isotridecyl myristate, ethyl laurate, N-methylpyrrolidone, ethyl oleate, oleic acid, isopropyl adipate, isopropyl palmitate, octyl palmitate, 1,3-butanediol and mixtures thereof.

In one embodiment, the hydrogel can be prepared with crosslinking monomers. Crosslinking monomers can include: dithiols; 3-[(2-aminoethyl)dithio]propionic acid-HCl-will react with an amine on one and a carboxylic acid on the other, bis-[beta-(4-azidosalicylamido)ethyl]disulfide-photoreactive phenylazides, dithiobis[succinimidyl propionate] (Lomant's reagent)-reactive succinimide will react with amines, cystamine, cystine, and homocystine-reactive coupling with carboxylic acids, dimethyl 3,3′-dithiobispropionimidate-2-HCl reactive toward amine groups, 3,3′dithiobis[sulfosuccinimidyl propionate] water soluble version of Lomant's reagent, ethylene glycol bis[succinimidyl succinate] and ethylene glycol bis[sulfosuccinimidyl succinate]-hydrolysis of ester groups, reactive toward amine functionality, (N-succinimidyl [4-azidophenyl]1,2′-dithiopropionate)-photoreactive and reactive toward amine, (sulfosuccinimidyl[4-azidophenyidithio]propionate)-water soluble, photoreactive and reactive toward amine dithiolglycolic acid and oxidized glutathione-reactive toward amines with carbodiimide assisted coupling, any H₂N—R—S—S—R′—NH₂-reactive toward carboxylic acids with carbodiimide. Also, crosslinking reagents can be employed to crosslink the polymers of the hydrogel after the polymers have been prepared into a hydrogel or pre-hydrogel composition.

In one embodiment, the hydrogel can include a disulfide cross linker. Such a hydrogel can be synthesized from polyacrylic acid in the presence of a crosslinker which has disulfide incorporated to make a crosslinked polymer with disulfide bonds. Such a hydrogel can be active as a bioadhesive and/or mucoadhesive hydrogel.

In one embodiment, the treatment agents can be physically trapped within the hydrogel, can be chemically attached to the hydrogel by crosslinking agents, or can hydrogen bond to the hydrogel, or any combination of physical and chemical attachment.

The treatment agent can be any compound or composition that provides a benefit to skin, which benefit can treat and/or prevent a skin condition, disease, disorder, or symptoms thereof, as well as maintain the skin in good condition. In order to identify a treatment agent for application in the skin care device, a target skin condition, disease, disorder, symptom, or maintenance need may be identified. As such, the treatment agent can be based on the treatment to be received by the skin.

Examples of skin conditions, disease, disorder, or symptoms thereof can include the following: acne, actinic keratosis, aged skin, angioma, athletes foot, aquagenic pruritus, argyria, atopic dermatitis, baldness, basal cell carcinoma, bed sores, Behcet's disease, blepharitis, boils, Bowen's disease, brittle skin, Bullous pemphigoid, canker sores, carbuncles, cellulitis, chloracne, condyloma acuminatum, chronic dermatitis, cold sores, contact dermatitis, cracked skin, creeping eruption, dandruff, dermatitis, dermatitis herpetiformis, dermatofibroma, diaper rash, dry skin, dyshidrosis, eczema, epidermolysis bullosa, erysipalas, erythrasma, erythroderma, friction blisters, favus, fungals, genital warts, gestational pemphigoid, Grover's disease, hemangioma, hidradentitis suppurativa, hives, Hodgkin's lymphoma hydrocystoma, hyperhidrosis, ichthyosis, impetigo, intertrigo, jock itch, Kaposi's sarcoma, keloid, keratocanthoma, keratosis pilaris, Lewandowsky-Lutz dysplasia, lice, lichen planus, lichen simplex chronicus, lipoma, loose skin, lyme disease, lymphadenitis, malignant melanoma, melasma, miliaria, molluscum contagiusum, mycosis fungoides, nummular dermatitis, otitis extema Paget's disease of the nipple, pediculosis, pemphigus, perioral dermatitis, photoallergy, photosensitivity, pityriasis rosea, pityriasis rubra pilaris, porphyria, psoriasis, pyoderma, Raynaud's disease, ringworm, rosacea, scabies, scaly skin, scleroderma, scrofula, sebaceous cysts, seborrheir keratosis, seborrheic dermatitis, shingles, skin cancer, skin tags, spider veins, squamous cell carcinoma, stasis dermatitis, sunburn, tick bites, tinea barbae, tinea capitis, tinea corporis, tinea cruris, tinea pedis, tinea unguium, tinea versicolor, tinea, tungiasis, urticaria, Vagabond's disease, vitiligo, warts, wheal, wrinkles, and any other skin condition or symptom thereof. Many of these conditions have well known treatments and treatment agents, and any of these treatment agents can be included in the hydrogel layer of the skin care device.

In order to treat the skin conditions, disease, disorder, or symptoms thereof as well as provide general maintenance, any of the following substances can be included in the hydrogel in an effective amount for treatment, prevention, and/or maintenance: accutane; adapalene; ajoene; alcohols (e.g., methanol, ethanol, etc.); albendazole; aloe vera; anesthetics (e.g., benzocaine, benzydamine, choline salicylate, prilocaine, lidocaine, tetracaine, etc.); amino acids (all amino acids and derivatives); antibiotics (e.g., dicloxacillin, cephalexin, erythromycin, flucloxacillin, phenoxymethylpenicillin, benzylpenicillin, penicillin, ampicillin amoxicillin, etc.); aspirin (acetylsalycylic acid); antiandrogen; antifungals (e.g., ketaconazole, ketoconazole, itraconazole, fluconzaole, naftifine, nystatin, caspofungin, allylamines, terminafine, amorolfine, butenafine, clotrimazole, sertaconazole, undecylenic acid); anti-inflammatories; antihistamines (e.g., diphenhydramine, cetirizine, etc.); antivirals (e.g., aciclovir, valaciclovir, famciclovir, penciclovir, herpevac, etc.); astringents (e.g., trichoroacetic acid, potassium hydroxide, cantharidin, podophyllin resin, etc.); azathioprine; azelaic acid; baking soda; benzoic acid; benzoyl peroxide; boric acid; cancer drugs; capsaicins; chemotherapeutics (e.g., adriamycin, bleomyin, vinblastine, dacarbazine, etc.); chlorhexidine gluconate; cimetidine; ciclosporin; clindamycin; colchicine; cortisone; corticosteroids (e.g., glucocorticoid, clobetasone butyrate, betamethasone valerate, triamcinolone, etc.); crocodile oil; cyproterone; dapsone; docosanol; doxycycline; drospirenone; erythromycin; etanercept; efalizumab; histamine blockers; H1 blockers; H2 blockers; hormones; hydrocortisone; hydroxychloroquine; ibuprofen; imiquimod; infliximab; interferons; isotretinoin; ivermectin; lamisil; lanolin; lindane; lymecycline; melaleuca oil; methotrexate; miconazole; minocycline; malathion; naproxen; nicotinamide; NSAIDs; onion extract; oestrogen; olestra; oxytetracycline; petroleum jelly; piroctone olamine; progestogen; potassium permanganate; permethrin; podophyllin; podofilox; rebamipide; rofecoxib; salicylic acid and derivatives; selenium sulfide; silver nitrate; spironolactone; stievamycin; sun block; tacrolimus; pimecrolimus; tazarotene; tetracycline; thalidomide; thiabendazole; topical retinoids; topical steroids; steroids; retinol; tea tree oil; terbinafine; tretinoin; triamcinolone acetonide; triclosan; tricloroacetic acid; trimethoprim; tromantadine; vitamin A; vitamin B; vitamin E; zinc; zinc oxide; zinc pyrithione; 5-fluorouracil; and other known or later developed agents.

Examples of some treatment agents for general skin maintenance can include: moisturizers; lotions; aloe vera; almond; avocado; carrot; castor; clay; cocoa; coconut oil; cornmeal; cucumber; cutch tree; emu oil; ginkgo biloba; ginseng; grape seed oil; ground almond and wallnut shell; horse chestnut; witch hazel; honey; lanolins; argan oil; vitamin D3; calcipotriol; retinoids; col tar; dithranol; sun screen; zinc oxide; titanium dioxide; oil in water emulsions; fragrances; allantoin; alpha lipoic acid; alum; ascorbic acid; bees wax; camphor; cetyl alcohol; cocoa butter; collagen; ecamsule; ellagic acid; ensulizole; free radical scavengers; glycerin; amino acids; homosalate; hydroquinone; lecithin; mineral oil; methyl gluceth; myristyl myristate; ococrylene; octyl salicylate; oxybenzone; PABA; panthenol; parabens; petrolatum; resveratrol; retinol; retinal; salicylic acid; silica; silicone; tinosorb; vitamins; nutrients; and other known or later developed agents for general skin maintenance. Any ingredient in a skin care composition can be formulated into the hydrogel layer of the skin care device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference in their entirety.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A device for delivering one or more treatment agents to skin, the device comprising:
a biocompatible hydrogel layer;
one or more treatment agents included in the hydrogel layer; and
a backing layer associated with the hydrogel layer

2. A device as in claim 1, wherein the biocompatible hydrogel layer is configured to retain structural integrity when in contact with skin.

3. A device as in claim 1, wherein the one or more treatment agents include a compound or composition configured to provide a skin treatment.

4. A device as in claim 1, wherein the backing layer is configured to retain structural integrity of the hydrogel layer when the hydrogel layer is in contact with the skin.

5. A device as in claim 1, wherein the one or more treatment agents are homogeneously dispersed within the hydrogel layer.

6. A device as in claim 1, wherein the one or more treatment agents form a concentration gradient in the hydrogel layer.

7. A device as in claim 1, wherein the one or more treatment agents are included in depots within the hydrogel layer.

8. A device as in claim 1, wherein the backing layer is aqueous liquid impermeable.

9. A device as in claim 1, wherein the backing layer is substantially hydrophobic.

10. A device as in claim 1, wherein the backing layer is paper.

11. A device as in claim 1, further comprising an adhesive layer between and coupling the backing layer to the hydrogel layer.

12. A device as in claim 1, further comprising a skin penetration enhancer included in the hydrogel layer, said skin penetration enhancer configured to enhance penetration of the one or more treatment agents into skin.

13. A device as in claim 1, wherein the one or more treatment agents are configured for treatment and/or prevention of at least one of acne, sunspots, skin cancer, wrinkles, dry skin, eczema, edema, psoriasis, rashes, shingles, infection, inflammation, itching, seborrheic dermatitis, atopic dermatitis, warts, rosacea, fungal infections, herpes, razor burn, mastocytosis, intertrigo, pityriasis rosea, lichen planus, hidradentis, symptoms thereof, or combinations thereof.

14. A device as in claim 1, further comprising a peelable layer associated with the hydrogel layer.

15. A device as in claim 1, wherein the hydrogel layer has a shape and size for application to a desired portion of skin of a subject.

16. A device as in claim 1, wherein the desired portion of skin is selected from the group consisting of a forehead, nose, cheek, lips, face, neck, shoulder, arm pit, arm, hand, finger, abdomen, chest, stomach, back, buttocks, genitals, legs, knee, feet, toes, and combinations thereof.

17. A device as in claim 12, wherein the shape and size is configured for application to a face of the subject.

18. A device as in claim 14, further comprising at least one opening in the hydrogel layer and backing layer that corresponds with at least one of an eye, nose, nostril, lips, mouth, or combination thereof.

19. A method of providing one or more treatment agents to skin of a subject, the method comprising:
providing a device configured for delivering the one or more treatment agents to the skin of the subject, the device including:
a biocompatible hydro gel layer:
one or more treatment agents included in the hydro gel layer; and
a backing layer associated with the hydrogel layer; and
applying the hydrogel layer of the device to the skin of the subject.

20. A method as in claim 19, further comprising peeling a peelable layer from the hydrogel layer so as to expose a surface of the hydrogel for application to the skin.

21. A method of manufacturing a device for delivering one or more treatment agents to skin, the method comprising:
preparing a biocompatible hydrogel or hydrogel-precursor composition;
introducing one or more treatment agents into the hydrogel or hydrogel-precursor composition; and
coupling a backing material to the hydrogel or hydrogel-precursor composition to form a backing layer.

22. A method as in claim 21, further comprising configuring the hydrogel layer to retain structural integrity when in contact with skin.

23. A method as in claim 21, wherein the backing layer is configured to retain structural integrity of the hydrogel layer when the hydrogel layer is in contact with skin.

24. A method as in claim 21, further comprising homogeneously dispersing the one or more treatment agents into the hydrogel or hydrogel-precursor composition.

25. A method as in claim 21, further comprising distributing the one or more treatment agents into the hydrogel layer in a concentration gradient.

26. A method as in claim 21, further comprising introducing the one or more treatment agents into the hydrogel layer as depots.

27. A method as in claim 21, further comprising disposing an adhesive layer between and coupling the backing layer to the hydrogel layer.

28. A method as in claim 21, further comprising introducing a skin penetration enhancer into the hydrogel layer, said skin penetration enhancer is configured to enhance penetration of the one or more treatment agents into skin.

29. A method as in claim 21, further comprising applying a peelable layer onto the hydrogel layer so as to protect the hydrogel layer from drying during storage.
